(19) United States
(12) Patent Application Publication
(10) Pub. No.: US 2006/0093570 A1
(43) Pub. Date: May 4, 2006

(54) Surface treatment

(76) Inventors: Andrea Duddington, Hull (GB); Paul Frederick Field, Hull (GB); David Norman Payne, Hull (GB)

Correspondence Address: NORRIS, MCLAUGHLIN & MARCUS, 875 THIRD AVE, 18TH FLOOR, NEW YORK, NY, 10022, US

(21) Appl. No.: 10/528,714

(22) PCT Filed: Oct. 10, 2003
(86) PCT No.: PCT/GB03/04381

§ 371 Date: Aug. 24, 2005

(30) Foreign Application Priority Data
Oct. 11, 2002 (GB) 0223570.3

(51) Int. Cl. A61K 31/19; A61K 8/36

(52) U.S. Cl. 424/70.22; 514/557

(57) ABSTRACT

This invention describes a surface treatment composition, preferably for personal use, comprising at least one surfactant and at least two different organic acids and/or salts or organic acids, and wherein the total concentration of the organic acids and/or salts in the composition is at least 0.5% (w/v). The invention further extends to a method of treating a surface, preferably hair or skin, to remove dirt and to remove or inhibit microbial growth.

This invention relates to surface treatment compositions, and methods of treating surfaces. The invention relates particularly, but not exclusively, to compositions for treatment of the skin or hair to effect cleaning, and to related methods. For the purpose of this specification the term “cleaning” denotes removal of dirt (including grease) and/or combating of microorganisms.

Many compositions used for surface treatment include a surfactant or combination of surfactants which help to release dirt and/or microorganisms from the hard surface. Such compositions may also contain biocidal agents. Such compositions must be formulated carefully. For example surfactants may denature biocidal agents. Many biocidal agents have a detectable odour and the type and amount of a fragrance—typically an expensive ingredient—may need to be carefully selected. Foaming qualities are usually desired and these can be compromised by subtle aspects of the formulation selection. The viscosity of the compositions can be hard to control. The desired viscosity may be reduced as a function of time, temperature or pH. Optimal action of a biocidal agent in the composition may require a particular pH range, whereas a different pH range may be needed for optimal viscosity.

The nature and combination of surfactants chosen will also contribute significantly to the physical properties of such a formulation. Consideration of the solubilisation of biocidal agents in such systems is crucial, to obtain and maintain efficacy.

Frequently, a compromise is reached in known compositions in which either the viscosity or biocidal efficacy of the composition, or both, is sacrificed in part, in order to provide a composition which provides at least reasonable viscosity and biocidal activity. In many known compositions, the pH of the composition is raised or lowered in order to optimise the viscosity, or a combination of surfactants is utilised to promote thermodynamic instability, in order to increase efficacy. In some compositions where viscosity is not compromised, biocidal activity is less than optimal.

It would therefore be advantageous to provide a surface treatment composition which provides a surfactant and biocidal action at optimal viscosity for good handling and high surfactant efficacy, but without reducing the efficacy of any biocidal agent(s) in the composition. It would also be advantageous to provide a composition which can be stored for relatively long periods of time without significant detrimental alteration of viscosity of the composition, or significant reduction in either surfactant effect or biocidal activity. Furthermore, it would be desirable to provide a surface treatment composition which includes a surfactant, the composition being at the optimum pH for good viscosity and viscosity maintenance, and good surfactant and biocidal action.

It is therefore an aim of preferred embodiments of the invention to overcome the problems of the prior art.

According to a first aspect of the present invention, there is provided a surface treatment composition comprising at least one surfactant and at least two compounds selected from organic acids and salts of organic acids, and wherein the total concentration of the organic acids and salts of organic acids in the composition is at least 0.5% (w/v).

Preferred acids for use in the present invention are carboxylic acids. Preferred salts of organic acids for use with the present invention are carboxylates, preferably alkali metal carboxylates, more preferably potassium or, especially, sodium salts.

Preferred carboxylic acids/carboxylates are aliphatic; especially saturated aliphatic.

Other suitable organic acids and salts thereof may include benzene sulphonic acid and other aromatic sulphonic acids, uric acid and other purine-containing acids and ascorbic acid and other sugar-derived acids.

Suitably one of the organic acids or salt of an organic acid has two or more carboxylic acid or carboxylate groups.

In a preferred embodiment, one of said compounds is an organic acid or a salt of an organic acid, having two or more carboxylic acid or carboxylate groups, and another of said compounds is an organic acid or a salt of an organic acid, having one carboxylic acid or carboxylate group.

In another preferred embodiment, one of said compounds is an organic acid or a salt of an organic acid, having three carboxylic acid or carboxylate groups, and another of said compounds is an organic acid or a salt of an organic acid, having one or two carboxylic acid or carboxylate groups.

Suitable organic acids/salts with one carboxylic acid or carboxylate group include linear or branched optionally substituted hydroxyalkyl carboxylic acids/salts or alkylmonocarboxylic acids/salts, of 1 to 8 chain carbon atoms, preferably 1 to 6 chain carbon atoms.

A suitable monocarboxylic acid/salt is formic, acetic, chloroacetic, dichloroacetic, benzoic, 2,4,6-trihydroxybenzoic, 2-aminobenzoic, pyruvic, quinolinic, 2-chlorobenzoic, glyoxylic, thioacetic, glyceric, acetoacetic, hippuric, glycolic acid, and especially, lactic acid; or salts thereof.

Suitable organic acids/salts with two carboxylic acids or carboxylate groups include linear or branched optionally substituted hydroxyalkyldicarboxylic acids/salts or alkyldicarboxylic acids/salts, of 2 to 10 chain carbon atoms, preferably 2 to 8 chain carbon atoms. Preferred organic dicarboxylic acids/salts include tartaric, oxalic, maleic, aspartic, L-glutamic, oxaloacetic, 2-oxogluteric, malonic, phthalic, methylmalonic, mesaconic, methylsuccinic, glutaric, malic and adipic acids or salts thereof.

Suitable organic acids/salts with three carboxylic acids or carboxylate groups include linear or branched optionally substituted hydroxyalkyltricarboxylic acids/salts, alkyltricarboxylic acids/salts, of 3 to 10 chain carbon atoms, preferably 3 to 8 chain carbon atoms. Preferred organic tricarboxylic acids/salts include L-argininosuccinic, isocitric and, especially, citric acid or salts thereof.

In a particularly preferred embodiment of the invention the composition comprises two organic acids and/or salts of organic acids; preferably carboxylic acids/salts. Preferably one of the organic acids/salts comprises lactic acid and/or lactate and another of the organic acids/salts comprises citric acid and/or citrate.

Preferably the composition comprises both a first organic acid and a salt of that organic acid.

Preferably the composition comprises both a second organic acid and a salt of that organic acid.

Suitably the total concentration of all of the organic acids and/or salts thereof in the composition is at least 1% (w/v), preferably at least 2% (w/v).

Suitably the total concentration of both organic acids and/or salts thereof in the composition is no more than 10% (w/v), preferably no more than 7.5% (w/v).

Suitably the pH of the composition is no more than 6, preferably no more than 5.5, more preferably no more than 5 and most preferably no more than 4.8.

Suitably the pH of the composition is at least 2, preferably at least 3 and more preferably at least 4.

One or, preferably, all of the organic acids and/or salts preferably act to buffer the composition to a desired pH.

The surfactant may be anionic, cationic, non-ionic, zwitterionic or amphoteric.

There may be more than one surfactant, preferably being independently selected from an anionic, cationic, non-ionic, zwitterionic or amphoteric surfactant.

Suitable non-ionic surfactants include alkoxylated alcohols, particularly alkoxylated fatty alcohols. These include ethoxylated and propoxylated fatty alcohols, as well as ethoxylated and propoxylated alkyl phenols, both having alkyl groups of from 7 to 16, more preferably 8 to 13 carbon chains in length.

Examples of alkoxylated alcohols include certain ethoxylated alcohol compositions presently commercially available from the Shell Oil Company (Houston, Tex.) under the general trade name NEODOL (trade mark), which are described as linear alcohol ethoxylates, certain compositions presently commercially available from the Union Carbide Company, (Danbury, Conn.) under the general trade name TERGITOL (trade mark) which are described as secondary alcohol ethoxylates, and contain compositions present commercially available from Clariant (UK) under the general trade name GENAPOL (trade mark) and which are described to be linear and branched alcohol ethoxylates.

Examples of alkoxylated alkyl phenols include certain compositions presently commercially available from the Rhône-Poulenc Company (Cranbury, N.J.) under the general trade name IGEPAL (trade mark), which are described as octyl and nonyl phenols.

Suitable anionic surfactants include linear C₈ to C₁₆ alkyl sulphates, C₈ to C₁₆ alkylsulphonates, C₈ to C₁₆ alkylbenzenesulphonates, C₈ to C₁₆ alkyldiphenyloxide disulphonates and C4 to C₁₋₆ alkylated naphthalene sulphonates. Suitable examples of anionic surfactants are sodium lauryl sulphonate and sodium dodecyl benezene sulphonate, or mixtures thereof. Preferably the anionic surfactant is selected from those comprising an alkali metal or ammonium cation.

A preferred composition of the present invention includes an anionic surfactant.

Suitable amphoteric surfactants include betaines.

A preferred composition of the present invention includes an amphoteric surfactant.

An especially preferred composition of the present invention includes an anionic surfactant in combination with an amphoteric surfactant. Preferably the ratio of the weight of the anionic surfactant to the weight of the amphoteric surfactant exceeds 1:1, and more preferably exceeds 2:1. Most preferably it exceeds 4:1. In highly preferred embodiments it exceeds 6:1.

Suitably the total concentration of the surfactant(s) in the composition is at least 2% (w/v), preferably at least 5% (w/v) and more preferably at least 8% (w/v).

Suitably the total concentration of the surfactant(s) in the composition is no more than 25% (w/v), preferably no more than 20% (w/v).

Suitably the composition is an aqueous composition. Preferably the composition comprises at least 50% (w/v) water, more preferably at least 60% (w/v), most preferably at least 70% (w/v).

The composition may comprise one or more further ingredients such as preservatives, thickeners, fragrance, chelating agents, and sodium chloride, for example.

The composition may contain a biocidal agent. The biocidal agent may be a bactericide, a viricide, a fungicide, a parasiticide, herbicide, algicide or any mixture of a combination thereof. Preferably it is a bactericide.

Suitably biocidal agents include phenolic compounds, such as PCMX.

There may be more than one biocidal agent present in the composition.

When a biocidal agent is present it may suitably be at a total concentration in the composition of at least 0.1% (w/v), preferably at least 0.2% (w/v) and more preferably at least 0.4% (w/v). Preferably it is present in an amount of up to 2% (w/v), more preferably up to 1% (w/v), most preferably up to 0.6% (w/v).

However it is believed that in preferred embodiments the acids and/or salts used in the invention may provide biocidal action, and it is possible that a traditional biocidal agent, such as an aromatic or heteroaromatic compound, notably a phenolic compound (for example PCMX), may not be needed in some embodiments. Accordingly compositions not containing such a biocidal agent are within the scope of the present invention.

Preferred compositions of the present invention have a foaming action with water on the surface to be treated.

According to a second aspect of the present invention there is provided a surface treatment composition comprising at least one surfactant and at least two different organic buffers.

Suitably the organic buffers comprise organic acids and salts thereof, as described above. Of course the buffers are selected to be compatible with each other in the composition, and compatible with other components of the composition.

Suitably the or each surfactant is as described for the first aspect of the invention.

Suitably the composition further comprises a biocidal agent as described for the first aspect of the invention.

Other definitions given above in relation the first aspect are applicable to the second aspect.

The composition of the first or second aspect is preferably a liquid skin cleaner (for example a hand wash), a shower gel, or the like.

In accordance with a third aspect of the present invention there is provided a package containing a composition of the first or second aspect, the package comprising a container for the composition and a restricted dispenser outlet therefrom under the control of a user. The restricted dispenser outlet could be, for example, a spray nozzle of a pressurised canister, or the outlet of a pump-action container, for example a press-action “tap” or the spray nozzle of a trigger spray container.

According to a fourth aspect of the invention there is provided a method of treating a surface, the method comprising the step of contacting the surface with the surface treatment composition of the first or second aspects of the invention.

Suitably the surface is a surface of a person, in particular the skin or hair of a person.

The method may comprise coating the surface with the composition, directly from a container or via the agency of a separate part, for example a sponge, cloth or the hand, or spraying the surface with the composition.

The method may comprise the final step of rinsing the surface with an aqueous media, suitably clean water.

### EXAMPLE

The various aspects of the invention will now be described with reference to the following non-limiting examples in which the following materials are used:

PCMX—parachloro meta-xylenol, supplied by Thomas Swan, Durham

EMPICOL ESB 70 (SLES)—sodium lauryl (C₁₂₋₁₆) ethoxy (2-3

EO)—sulphate surfactant, supplied by Huntsman

EMPIGEN BSFA—a betaine amphoteric surfactant, supplied by Huntsman

KATHON CG—a preservative; a mixture of thiazolinones, supplied by Rohn & Haas

JAGUAR EXCEL—a guar gum supplied by Rhodia

Pine fragrance

EMPICOL XPE/H—pearliser, supplied by Huntsman

EMPICOL, EMPIGEN, KATHON and JAGUAR EXCEL are trade marks.

A composition of the invention was made up according to Formulation A given in Table 1 below, in which lactic acid/sodium lactate and citric acid/sodium citrate were used as two different buffering agents. A second formulation, Formulation B was also prepared in which the citric acid/sodium citrate was omitted. A control formulation, Formulation C was prepared in which no such organic acid or salt was present.

TABLE 1

| | Concentration (% w/v) | | |
|---|---|---|---|
| Ingredient | Formulation A | Formulation B | Formulation C |
| PCMX | 0.5 | 0.5 | 0.5 |
| EMPICOL ESB 70 | 9.0 | 9.0 | 9.0 |
| EMPIGEN BSFA | 1.5 | 1.5 | 1.5 |
| Lactic acid | To pH 4.7 | To pH 4.7 | — |
| Sodium lactate | 1.0 | 1.0 | — |
| EMPICOL XPE/H | 1.5 | 1.5 | 1.5 |
| Fragrance | 0.2 | 0.2 | 0.2 |
| KATHON CG | 0.02 | 0.02 | 0.02 |
| Tetrasodium EDTA | 0.3 | 0.3 | 0.3 |
| Sodium citrate | 0.7 | — | — |
| Citric acid | To pH 4.7 | — | — |
| JAGUAR EXCEL | 0.3 | 0.3 | 0.3 |
| Sodium chloride | — | — | Q.S |
| Deionised water | to 100% | to 100% | to 100% |

The anti-microbial efficacy of Formulation A, Formulation B and Formulation C against Staphylococcus aureus (NCTC 10788) and Escherichia coli (NCTC 10418), was tested by performing a Handwash Efficacy Suspension Test.

The Handwash Efficacy Suspension Test is based on a standard test for the assessment of the rapid germicidal activity for antibacterial liquid and bar soap products, test prEN12054—chemical disinfection and antiseptics—Products for hygienic and surgical handrub and handwash, bactericidal activity, test method and requirements (phase 2, step 1); British Standard Institute draft for public comment 95/561926 July 1995; but with use of a different E. coli strain).

The microbiocidal effect (ME) due to the action of the composition over the test contact time at the temperature at which the test was performed is expressed by the formula:
ME=Log N_{C}−Log N_{D}
where:

N_{C}=Number of cfu/ml of the relevant control test (test mixture without composition).

N_{D}=Number of cfu/ml of the test mixture after the action of the composition.

Results are graded as follows:

| ME values obtained | Activity |
|---|---|
| >4.0 | Excellent |
| 3.0-4.0 | Good |
| 1.5-3.0 | Moderate |
| 0.5-1.5 | Poor |
| <0.5 | No activity |

Formulations A, B and C were diluted in hard water to give a 50% v/v concentration, and were tested against S. aureus by contacting Formulations A, B and C with S. aureus cultures for one minute.

The tests were repeated a further two times to give a total of three repeats.

The results of the anti-microbial efficacy test against S. aureus are shown in Table 2.

TABLE 2

| | Replicate ME Values | | | Median ME |
|---|---|---|---|---|
| Formulation | Run 1 | Run 2 | Run 3 | Value |
| A | 2.68 | 2.48 | 3.08 | 2.68 |
| B | 0.95 | 0.78 | 2.23 | 0.95 |
| C | 1.38 | 1.25 | 1.61 | 1.38 |

The anti-microbial efficacy of Formulations A, B and C was tested against Escherichia coli, using the Handwash Efficacy Suspension Test as detailed above. The results of the test against E. coli) is shown in Table 3 below.

TABLE 3

| | Replicate ME Values | | | Median ME |
|---|---|---|---|---|
| Formulation | Run 1 | Run 2 | Run 3 | Value |
| A | 4.68 | 4.95 | 4.57 | 4.68 |
| B | 2.24 | 2.60 | 2.60 | 2.60 |
| C | 0.24 | 0.09 | 0.17 | 0.17 |

The results of the test as shown in Tables 2 and 3 show that Formulation A exhibited moderate activity against S. aureus with a median ME value of 2.68.

Formulation A also showed good activity against E. coli achieving a median ME value of 4.68. Formulation B showed moderate activity against E. coli compared to Formulation A, with median ME value of 2.6, whereas Formulation C without organic buffers showed no activity against this organism.

Viscosity stability issues were also studied. The polymeric thickener CROTHIX was found to be unstable at the pH of Formulation B (pH 4.7). The pH of the formulation was increased to pH 5.2 to avoid viscosity degradation over time. The microbial efficacy of the product decreased considerably as compared to Formulation B at pH 4.7. Also, after storage at 50° C. for two weeks, the pH 5.2 formulation exhibited significant viscosity degradation. It was noticed that pH decreased over this time period. As such, an increase in buffering capacity of Formulation B was investigated by increasing the amount of sodium lactate/lactic acid pairing, which resulted in excess of sodium ions, and as a result, the formulation was not capable of thickening.

Conversely, Formulation A, employing namely sodium citrate/citric acid and sodium lactate/lactic acid, counteracted the loss of thickening capacity at pH 5.2, such that Formulation A at pH 5.2 showed significantly decreased viscosity degradation over time, with no significant loss of biocidal effect, compared to Formulation B in which the pH was increased by 5.2 by addition of further sodium lactate/lactic acid.

The biocidal efficacy of Formulation A at pH 5.2 was much higher (5 log reductions v. S. aureus and E. coli) compared to Formulation B at pH 5.2.

In further tests of a corresponding composition containing the citric and lactic buffer pairs bur not containing PCMX nor any other accepted biocidal agent, significant biocidal activity was still obtained.

1. A hard surface cleaning concentrate composition comprising:
a) at least one non-cationic antimicrobial agent;
b) at least one solvent selected from water soluble organic solvent, water insoluble organic solvent, terpenes, essential oil, and mixtures thereof;
c) an anionic soap surfactant;
d) at least one surfactant selected from nonionic surfactant, anionic surfactant excluding the anionic soap of c), and mixtures thereof;
e) optionally, one or more alkanolamines;
f) optionally, one or more conventional constituents selected from dyes, colorants, fragrances and fragrance solubilizers/enhancers, light stabilizers, viscosity modifying agents, pH adjusting agents and pH buffers including organic and inorganic salts, optical brighteners, opacifying agents, hydrotropes, antifoaming agents, enzymes, anti-spotting agents, anti-oxidants, preservatives, and anti-corrosion agents; and
g) the balance, water
characterized in that the concentrate compositions are mixed with water in dilution of 1 part concentrate composition to 50-200 parts water at 20° C., the resultant mixture exhibits a light transmittance loss of at least 30%.

2. The cleaning concentrate according to claim 1 wherein the a) non-cationic antimicrobial agent is selected from pyrithiones, dimethyldimethylol hydantoin, methylchloroisothiazolinone/methylisothiazolinone sodium sulfite, sodium bisulfite, imidazolidinyl urea, diazolidinyl urea, benzyl alcohol, 2-bromo-2-nitropropane-1,3-diol, formal in (formaldehyde), iodopropenyl butylcarbamate, chloroacetamide, methanamine, methyldibromonitrile glutaronitrile, glutaraldehyde, 5-bromo-5-nitro-1,3-dioxane, phenethyl alcohol, o-phenylphenol/sodium o-phenylphenol, sodium hydroxymethylglycinate, polymethoxy bicyclic oxazolidine, dimethoxane, thimersal dichlorobenzyl alcohol, captan, chlorphenenesin, dichlorophene, chlorbutanol, glyceryl laurate, halogenated diphenyl ethers, phenolic compounds, mono- and poly-alkyl and aromatic halophenols, resorcinol and its derivatives, bisphenolic compounds, benzoic esters (parabens), halogenated carbanilides, 3-trifluoromethyl-4,4-dichlorocarbanilide, and 3,3,4-trichlorocarbanilide.

3. The cleaning concentrate according to claim 1 wherein the a) non-cationic antimicrobial agent is a mono- and poly-alkyl and aromatic halophenol selected from the group p-chlorophenol, methyl p-chlorophenol, ethyl p-chlorophenol, n-propyl p-chlorophenol, n-butyl p-chlorophenol, n-amyl p-chlorophenol, sec-amyl p-chlorophenol, n-hexyl p-chlorophenol, cyclohexyl p-chlorophenol, n-heptyl p-chlorophenol, n-octyl p-chlorophenol, o-chlorophenol, methyl o-chlorophenol, ethyl o-chlorophenol, n-propyl o-chlorophenol, n-butyl o-chlorophenol, n-amyl o-chlorophenol, tert-amyl o-chlorophenol, n-hexyl o-chlorophenol, n-heptyl o-chlorophenol, o-benzyl p-chlorophenol, o-benzyl-m-methyl p-chlorophenol, o-benzyl-m, m-dimethyl p-chlorophenol, o-phenylethyl p-chlorophenol, o-phenylethyl-m-methyl p-chlorophenol, 3-methyl p-chlorophenol, 3,5-dimethyl p-chlorophenol, 6-ethyl-3-methyl p-chlorophenol, 6-n-propyl-3-methyl p-chlorophenol, 6-iso-propyl-3-methyl p-chlorophenol, 2-ethyl-3,5-dimethyl p-chlorophenol, 6-sec-butyl-3-methyl p-chlorophenol, 2-iso-propyl-3,5-dimethyl p-chlorophenol, 6-diethylmethyl-3-methyl p-chlorophenol, 6-iso-propyl-2-ethyl-3-methyl p-chlorophenol, 2-sec-amyl-3,5-dimethyl p-chlorophenol 2-diethylmethyl-3,5-dimethyl p-chlorophenol, 6-sec-octyl-3-methyl p-chlorophenol, p-chloro-m-cresol, p-bromophenol, methyl p-bromophenol, ethyl p-bromophenol, n-propyl p-bromophenol, n-butyl p-bromophenol, n-amyl p-bromophenol, sec-amyl p-bromophenol, n-hexyl p-bromophenol, cyclohexyl p-bromophenol, o-bromophenol, tert-amyl o-bromophenol, n-hexyl o-bromophenol, n-propyl-m,m-dimethyl o-bromophenol, 2-phenyl phenol, 4-chloro-2-methyl phenol, 4-chloro-3-methyl phenol, 4-chloro-3,5-dimethyl phenol, 2,4-dichloro-3,5-dimethylphenol, 3,4,5,6-terabromo-2-methylphenol, 5-methyl-2-pentylphenol, 4-isopropyl-methylphenol, para-chloro-meta-xylenol, dichloro meta xylenol, chlorothymol, and 5-chloro-2-hydroxydiphenylmethane.

4. The composition according to claim 1 wherein the b) solvent is selected from C₁₋₄ alcohols, terpenes, essential oil, and mixtures thereof.

5. The composition according to claim 4 wherein the b) solvent is a mixture of essential oil and C₁₋₄ alcohol.

6. (canceled)

7. (canceled)

8. (canceled)

9. (canceled)

10. The composition according to claim 5 wherein the b) solvent is a mixture of essential oil and ethanol.

11. The composition according to claim 10 wherein the essential oil is a mixture of pine oil and d-limonene.

12. (canceled)

13. The composition according to claim 1 wherein the anionic soap surfactant is selected from alkali metal soap fatty acids.

14. The composition according to claim 1 wherein the non-cationic antimicrobial agent is present in an amount of from about 0.05 to about 15 wt %.

15. The composition according to claim 1 wherein the anionic soap surfactant is present in an amount of from about 0.1 to about 20 wt %.

16. The composition according to claim 1 wherein the d) surfactant is a mixture of nonionic surfactant and anionic surfactant excluding the anionic soap of c).

17. The composition according to claim 1 wherein the d) surfactant is nonionic surfactant.

18. The composition according to claim 1 wherein the d) surfactant is an anionic surfactant excluding the anionic soap of c).

19. The composition according to claims 16 and 17 wherein the nonionic surfactant is an alcohol ethoxylate or an alkylphenol ethoxylate.

20. (canceled)

21. The composition according to claim 16 wherein the anionic surfactant excluding the anionic soap of c) is a sulfate or sulfonate.

22. (canceled)

23. (canceled)

24. The composition according to claim 1 wherein the d) surfactant is present in an amount of from about 0.01 to about 10 wt %.

25. The composition according to claim 1 which contains e) at least one alkanolamine.

26. The composition according to claim 25 wherein the alkanolamine is monoethanolamine.

27. A hard surface cleaning concentrate composition comprising:
a) from about 0.05 to about 15 wt %, of at least non-cationic antimicrobial agent;
b) from about 0.1 to about 20 wt %, of at least one solvent selected from water soluble organic solvent, water insoluble organic solvent, terpene, essential oil, and mixtures thereof;
c) from about 0.1 to about 20 wt %, of an anionic soap surfactant;
d) from about 0.01 to about 10 wt %, of at least one surfactant selected from nonionic surfactant, anionic surfactant excluding the anionic soap of c), and mixtures thereof;
e) from about 0.1 to about 10 wt % of one or more alkanolamines;
f) from about 0 to about 10 wt % of one or more conventional constituents selected from dyes, colorants, fragrances and fragrance solubilizers/enhancers, light stabilizers, viscosity modifying agents, pH adjusting agents and pH buffers including organic and inorganic salts, optical brighteners, opacifying agents, hydrotropes, antifoaming agents, enzymes, anti-spotting agents, anti-oxidants, preservatives, and anti-corrosion agents; and
g) the balance, water
characterized in that the concentrate compositions are mixed with water in dilution of 1 part concentrate composition to 50-200 parts water at 20° C., the resultant mixture exhibits a light transmittance loss of at least 30%.

28. The composition according to claim 27 which contains e) one or more alkanolamines.

29. (canceled)

30. A process for cleaning and/or disinfecting a hard surface requiring such treatment which process includes the steps of:
dispersing in water in a weight ratio of concentrate composition:water of from 1:0.1 to 1:1000 a composition according to claim 1; and
applying the dispersed concentrate to the hard surface in an amount effective for providing cleaning and/or disinfecting treatment of the hard surface.
